(19) United States
(12) Patent Application Publication
(10) Pub. No.: US 2025/0288246 A1
(43) Pub. Date: Sep. 18, 2025

(54) Method for determining a scale for the degree of obstructive sleep apnoea and/or the after-effect thereof, such as daytime sleepiness

(71) Applicant: Universitätsmedizin der Johannes Gutenberg-Universität Mainz, Mainz (DE)

(72) Inventors: Haralampos Gouveris, Mainz (DE); Muthuraman Muthuraman, Freimersheim (DE)

(21) Appl. No.: 18/857,910

(22) PCT Filed: Apr. 19, 2023
(86) PCT No.: PCT/EP2023/060099

§ 371 Date: Jun. 10, 2025

(30) Foreign Application Priority Data
Apr. 27, 2022 (EP) 22170323.4
Jan. 19, 2023 (EP) 23152373.9

(51) Int. Cl. A61B 5/00; A61B 5/256; A61B 5/374; G16H 50/20; G16H 50/30

(52) U.S. Cl. CPC A61B 5/4818; A61B 5/256; A61B 5/374; A61B 5/4812; A61B 5/6803; A61B 5/7275; G16H 50/20; G16H 50/30; A61B 2560/0468

(57) ABSTRACT

The present invention relates to a method for determining the measure of the degree of an obstructive sleep apnea and/or its consequence by means of the following steps:
defining a measure of the degree of an obstructive sleep apnea and/or its consequence,
providing two EEG measurement signals of an electroencephalography at the electroencephalography points of an 10-20 international EEG system,
dividing the EEG measurement signals into frequency bands,
determining at least one cross-frequency modulation index using data from at least two different frequency bands,
determining the measure of the degree of an obstructive sleep apnea and/or its consequences by means of the at least one cross-frequency modulation index.

The invention further relates to a device for carrying out the method.

### CROSS REFERENCE TO RELATED APPLICATIONS

This is a national stage filing in accordance with 35 U.S.C. § 371 of PCT/EP2023/060099, filed Apr. 19, 2023, which claims the benefit of the priority of European Patent Application No. 22170323.4, filed Apr. 27, 2022 and European Patent Application No. 23152373.9, filed Jan. 19, 2023, the contents of each are incorporated herein by reference.

### DESCRIPTION

Method for determining a measure of the degree of an obstructive sleep apnea and/or its consequence, such as daytime sleepiness

### TECHNICAL FIELD

The present invention relates to a method for determining a measure of the degree of an obstructive sleep apnea and/or its consequence, such as daytime sleepiness.

### STATE OF THE ART

Sleep apnea is a nocturnal respiratory disorder, in which prolonged or partial respiratory arrests repeatedly occur during sleep. By definition, prolonged respiratory arrests or prolonged partial respiratory arrests are respiratory arrest or partial respiratory arrest that last longer than 10 seconds. Short respiratory arrests lasting less than 10 seconds are usually not considered health-damaging. However, if longer respiratory arrests or partial respiratory arrests occur more than five times per hour during sleep, severe health-damaging consequences are possible.

Obstructive sleep apnea (OSA) is common in adults. The disorder can cause neuronal damage or OSA itself can be a neurological disorder. Stimulation of the hypoglossal nerve, which is an effective treatment for many OSA patients, supports the argument that OSA is a neuronal/neuromuscular disorder. Accordingly, evidence of a separation between motor-associated cortical neuronal groups and peripheral upper cervical motor units has been found in OSA. Furthermore, there is evidence from functional MRT studies for a separation between sensorimotor and other cortical regions in OSA.

Sleepiness, a commonly reported effect of OSA, has significant associations with functional connectivity within the sensorimotor network. A functional neuroimaging study in adults over 55 years showed a hypoperfusion in sensorimotor areas of patients with severe OSA. Furthermore, neuroimaging studies of interhemispheric interaction observed a strong correlation between sleepiness and the activity of the bilateral precentral gyrus, an important part of the sensorimotor network, after sleep deprivation.

Sleep apnea or the severity of sleep apnea is usually diagnosed in a sleep laboratory by means of a cardiorespiratory polysomnography. The severity is currently divided into three groups of severity.

Measurement sensors are attached to the patient suffering from obstructive sleep apnea for one or two consecutive nights. For approximately 8 hours, 18 different physiological signals are recorded: electroencephalogram (EEG, 4 signals), electrooculogram (EOG. 2 signals), electromyogram (EMG, 3 signals) on the chin and both lower legs, electrocardiogram (EKG, 1 signal), measuring the pulse rate and oxygen saturation in the blood (pulse oximetry, 2 signals), respiratory flow measurement via the nose and mouth (2 signals), respiratory effort on the abdomen and chest (2 signals), snoring microphone on the neck (1 signal) and body position (1 signal).

As part of cardiorespiratory polysomnography (KRPSG), the severity of OSA is calculated using the RDI (Respiratory Disturbance Index), i.e., the number of apneas, hypopneas, and so-called RERAs (respiratory effort-related arousals) per hour of sleep. People with RDI >15/h of sleep have clinically significant OSA and should be treated soon according to current guidelines. People with RDI=5-15/h have mild OSA and should not necessarily be treated soon and people with RDI <5/h are healthy (do not suffer from OSA).

The Epworth Sleepiness Scale (EES) is used for the subjective assessment of daytime sleepiness in patients with OSA (Johns 1991). It is a questionnaire with 8 questions for the subjective (sell-perceived) assessment of the tendency of daytime sleepiness in monotonous everyday situations (no tendency=0 points; maximum tendency=3 points; i.e. the cumulative score can vary between 0 and 24 points). With a total EES score >10, the probability of significant, clinically relevant daytime sleepiness is very high.

The measurement is then evaluated automatically or manually using the algorithms embedded in the commercially available KRPSG software systems, which use eight of the 18 KRPSG signals mentioned above to calculate the RDI. For quality assurance, it is therefore essential that a visual evaluation is carried out by an expert (caregiver, medical-technical assistant, doctor), in each case with a varying level of education.

To calculate the EES, a subjective assessment of daytime sleepiness using the ESS questionnaire is necessary.

This method is comparatively complex.

Representation of the Invention:

It is object of the present invention to provide a preferably automated method for recording and particularly for evaluating measurement data, which makes it possible to easily make a statement on the basis of the data about a measure of the severity of OSA and the extent of the associated subjectively assessed daytime sleepiness in OSA patients in a simple manner, particularly to be able to easily make a corresponding statement without the involvement of experts.

This object is solved by a method for determining a measure of the degree of an obstructive sleep apnea and/or its consequence by means of an evaluation of measurement data according to claim 1, in which first a measure of the degree of an obstructive sleep apnea and/or its consequence is determined, and then two EEG measurement signals of an electroencephalography are provided at the electroencephalography points of a 10-20 international EEG system. The EEG measurement signals are divided into frequency bands, in particular filtered accordingly. At least one cross-frequency modulation index is determined from the data of at least two different frequency bands. The measure of the degree of an obstructive sleep apnea and/or its consequence is then determined using the at least one cross-frequency modulation index.

In this method, only two EEG signals are therefore provided and evaluated. The claimed method generates data suitable for a correlation with a measure of the degree of an obstructive sleep apnea and/or its consequence, so that a statement can be made about the degree of obstructive sleep apnea and/or its consequence. The provision and evaluation of the data can be fully automated.

One measure for the severity of obstructive sleep apnea is, for example, the Respiratory Disturbance Index. One consequence of obstructive sleep apnea is daytime sleepiness. The degree of daytime sleepiness is indicated using the Epworth Sleepiness Scale (ESS), for example.

The 10-20 international EEG system is an internationally recognized method for describing the position of scalp electrodes as part of an EEG measurement, particularly a polysomnography sleep study.

It is particularly advantageous if the EEG measurement signals are recorded at home during sleep. However, it is also possible to record the data in a sleep laboratory or a doctor's office.

The method is particularly easy to implement if, according to a preferred embodiment, the measure of the degree of an obstructive sleep apnea and/or its consequence, particularly the Respiratory Disturbance Index and/or daytime sleepiness, is determined only on the basis of the two EEG measurement signals.

It has proven to be particularly advantageous that the electroencephalography points at which the EEG measuring signals are recorded are the points C3 and C4. The measurement data obtained at these points can be used to determine the Respiratory Disturbance Index or a measure of daytime sleepiness with comparatively high accuracy.

It is an advantage that the measurement signals are divided into frequency bands separately for each measurement point in order to exclude measurement interference at one measurement point.

The invention is based on the following observations:

Cross-frequency coupling (CFC) is a fundamental feature of brain oscillatory activity and correlates strongly with brain function. CFC comprises different patterns: phase synchronization; amplitude co-modulation and phase-amplitude coupling (PAC). In the present invention, PAC were analyzed as it represents neuronal coding and information transmission within local microscale and macroscale neuronal ensembles of the brain. Low-frequency oscillatory activity represents the regulation of the information flow between brain areas by modulating the excitability of local brain ensembles. The phase of this low-frequency oscillatory activity influences both the rhythm of high-frequency activity and the firing rate of individual neurons. Thus, phase-amplitude cross-frequency coupling (PACFC) appears to promote effective interaction between neurons of similar phase preferences and synchronization of high-frequency bands during specific slower rhythmic phases.

Understanding neurophysiological connectivity patterns and oscillatory dynamics at the sensorimotor cortical areas can help to further clarify the pathophysiological processes of the central nervous system (CNS) in OSA. Due to the fact that OSA is clinically associated with cognitive, alertness and wakefulness disorders during wakefulness (particularly excessive daytime sleepiness), a better understanding and modeling of such connectivity can provide the basis for predicting relevant clinical phenomena.

This invention characterizes the functional network connectivity of the sensorimotor area in therapy-naïve OSA patients. For this purpose, it has been initially tested whether sleep stage-specific theta-gamma PACFC modulation differs between patient with and without significant OSA. This assessment was based on the results of previous studies that have shown a significant role of theta oscillation during different sleep stages. Furthermore, it was analyzed whether these modulations were specific to a particular sleep stage. To assess whether a possible functional separation in the sensorimotor area was frequency-specific, the delta-alpha PACFC modulation was compared between patients with and without significant OSA as control experiment. Finally, it was analyzed whether such a separation was correlated with clinical parameters such as patient-reported sleepiness results (Epworth Sleepiness Scale; EES).

Prior studies have used cross-frequency coupling for the classification of sleep stages, particularly in healthy adults, but also in OSA patients. However, none of these studies assessed the significance of this classification in regard to clinical applicability for OSA patients. In this invention, the frequency-specific CFC-based modulation indices are not only used for the classification of sleep stages, but further show that the same modulation indices could also predict clinical scores like ESS. Thus, these results should help to solve whether there is either a frequency-specific, sleep stage-specific or global functional separation in the sensorimotor area of OSA patients. Furthermore, this can provide an objective, neurophysiological surrogate marker to quantify patient-reported subjective sleepiness as well as the severity of respiratory disease in OSA patients.

In a preferred embodiment of the method, the at least one cross-frequency modulation index is therefore determined by means of phase-amplitude cross-frequency coupling. For this purpose, the measurement signals are preferably divided into at least two of the following frequency bands: low frequency band from 0.1. to 1 Hz, delta band (1 to 3 Hz), theta band (4 to 7 Hz), alpha band (8-13 Hz), beta band (14 to 30 Hz) and gamma band (31-100 Hz) (31-100 Hz).

In particular, the two measurement signals are divided into the two frequency bands alpha band (8-13 Hz) and delta band (1 to 3 Hz), wherein the amplitude envelope is preferably determined from the alpha band and the phase of the measurement signals is determined from the delta band by means of phase-amplitude cross-frequency coupling.

Additionally, or alternatively, the measurement signals are divided into the two frequency bands theta band (4 to 7 Hz) and gamma band (31-100 Hz), wherein the amplitude envelope is preferably determined from the gamma band and the phase of the measurement signals is determined from the theta band by means of phase-amplitude cross-frequency coupling.

The EEG measurement signals required for the method are preferably stored in a database or a memory module so that they are available independent of the time and location of the recording of the EEG measurement signals. This makes it possible, for example, to create a correlation database in which a measure of the degree of an obstructive sleep apnea and/or its consequence is correlated with the cross-frequency modulation index.

In a preferred embodiment, a correlation database with correlation data between the at least one cross-frequency modulation index and the measure of the degree of an obstructive sleep apnea and/or its consequence is provided, wherein the measure of the degree of an obstructive sleep apnea and/or its consequence is then determined from the at least one cross-frequency modulation index using the data of the correlation database.

Providing a correlation database enables a particularly simple and precise assignment of the cross-frequency modulation index to a measure of the degree of an obstructive sleep apnea and/or its consequence.

Preferably, the measure of the degree of an obstructive sleep apnea and/or its consequence, particularly the Respiratory Disturbance Index and/or daytime sleepiness, is determined by means of a Support Vector Machine on the basis of the at least one cross-frequency modulation index.

In summary, phase-amplitude cross-frequency coupling indicates the modulation of the high-frequency power in the C3/C4 EEG signal by the low frequency phase in the same (C3/C4) EEG signal. This cross-frequency coupling (CFC) modulation index (MI) is then used to identify the phase-amplitude assignment between the phase-modulating frequency bands (e.g., delta) and the amplitude-modifying frequency bands (e.g., gamma). For the measurement of the cross-frequency coupling measure, only the signals recorded by the brain, i.e., signals from the C3 and C4 electrodes, are used. After estimating the MI, the Support Vector Machine learning algorithm is used to predict the RDI as well as the ESS (Epworth Sleepiness Scale).

In a preferred embodiment of the method described, the measurement signals are recorded during sleep and the sleep is divided into sleep stages, wherein the at least one cross-frequency modulation index is determined depending on the sleep stages, so that an assignment of the cross-frequency modulation index and the measure of the degree of an obstructive sleep apnea and/or its consequence can be indicated depending on the sleep stages. This makes it possible to make very precise statements about the severity of sleep apnea and daytime sleepiness, as it has been observed that the individual cross-frequency modulation indices differ in the individual sleep stages depending on the severity of sleep apnea and/or daytime sleepiness.

It is particularly an advantage that all method steps are carried out automatically, wherein an automated classification of sleep stages is advantageously carried out when the measurement signals are recorded during sleep.

Another object of the present is a device for carrying out a method for determining a measure of the degree of an obstructive sleep apnea and/or its consequence, particularly a device for carrying out the method described, wherein the device comprises a headgear with two sensors for recording EEG signals, particularly at the points C3 and C4.

Since only two measurement points are required to carry out the method according to the invention, it is sufficient that the headgear comprises only two sensors for determining the EEG signals, particularly at the points C3 and C4.

A data memory and a data processing module can also be provided. The data memory is provided to store the EEG signals and make them available for further evaluation. The data processing module is provided to determine a cross-frequency modulation index from the data according to the present invention. The data processing module is further designed to determine a measure of the degree of an obstructive sleep apnea and/or its consequence from the cross-frequency modulation index.

In a preferred embodiment, the headgear is a beanie, a cap, wherein the cap can also have a chin part, or a headband.

The invention has the advantage that RDI and the ESS are determined using only two EEG electrodes (instead of the standard eight of the 18 electrodes mentioned above) in patients with OSA. The method is thus less complex and less expensive than known methods.

Due to the small amount of different measurement signals, a corresponding device for carrying out the method can be designed in a way that it is less disturbing for the test subject.

This is particularly advantageous for measurements during sleep, as the sleep is not disturbed or significantly less disturbed compared to already known devices due to the measurement device for recording measurement signals. Furthermore, both the RDI and the ESS are determined fully automatically by means of the invention, so that an objective method for determining/predicting daytime sleepiness is available.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Preferred embodiments of the invention are described in more detail by means of the attached drawings, in which show:

FIG. 1 an overview of the data acquisition and data analysis process.

FIG. 2A the MI differences in theta-gamma CFCs, main dataset.

FIG. 2B the MI differences in theta-gamma CFCs, validation dataset,

FIG. 3A the MI differences in delta-alpha CFCs, main dataset,

FIG. 3B the MI differences in delta-alpha CFCs, validation dataset,

FIG. 4 the SVM classification of different sleep stages,

FIG. 5 the SVM prediction of RDI and ESS,

FIG. 6 the correlation of clinical parameters with CFC measurements,

FIG. 7 the subsequent distribution of the analyzed groups, and

FIG. 8 a measurement device for recording the EEG signals C3 C4.

FIG. 9 a first alternative embodiment of a measurement device for recording the EEG signals C3 C4, and

FIG. 10 a second alternative embodiment of a measurement device for recording the EEG signals C3 C4.

### Ways of Carrying Out the Invention and Industrial Applicability:

A total of 170 participants were included in a study conducted using the method according to the invention. The patients were divided into main and validation datasets with 86 participants in the main dataset: 22 women, age group: 27-84 years, 44 subjects with moderate or severe OSA and 84 participants in the validation dataset: 28 women, age group 35-75 years, 42 subjects with moderate or severe OSA. The data used for the analysis were evaluated retrospectively. Consecutive datasets of patients after application of the inclusion and exclusion criteria mentioned below were included for analysis. Therefore, a retrospective non-randomized case-control study design was used. All patients had initially visited the clinic of the Sleep Medicine Center of an academic medical center due to complaints about snoring and/or daytime sleepiness. All participants were initially diagnosed with OSA based on the PSG recordings used in this study. Thus, all participants were unexperienced in therapy and did not have positive airway pressure therapy or upper airway surgery or therapy with any mandible advancement devices used previously.

Inclusion and exclusion criteria for participants were based on conditions that could have influenced the development and the severity of observed OSA and/or EEG recordings. Data from adult patients (aged ≥18 years) who complained about snoring and/or daytime sleepiness in the clinic, had no previous therapy for sleep-related respiratory disorders, and were subjected to nocturnal polysomnography at our sleep medicine center were included. Participants with neurodegenerative (e.g., Parkinson's disease) or neuroinflammatory (e.g., multiple sclerosis) diseases, history of stroke, heart failure based on the New York Heart Association (NYHA)—stages 3 or 4, chronic obstructive pulmonary disease (COPD), all psychiatric disorders were excluded from the study. Furthermore, subjects regularly using sedatives, benzodiazepines, serotonin reuptake inhibitors or other psychotropic medication, malignant diseases of any kind, radiation therapy of an anatomical cranial or cervical region, surgery on intracranial structures, or surgery for the therapy of sleep-related respiratory disorders (either snoring or OSA) were further excluded from the study. Patient-reported results of excessive daytime sleepiness (EDS) were recorded using the Epworth Sleepiness Scale (ESS).

All-night polysomnography (PSG) recordings from 170 thoroughly examined subjects, who met the study entry criteria were used for analysis. All participants were subjected to an overnight polysomnography (PSG) with recordings of the electroencephalogram, electrooculogram, submental and pretibial electromyogram, and electrocardiogram. Polysomnography (PSG) was recorded according to current AASM (American Academy Sleep Medicine, Inc.) standards to determine the type and degree of sleep-related respiratory disorders. Nasal air flow was visualized by measuring the impact pressure using a nasal senor in which pressure oscillations of the respiratory airflow were determined. Thoracic and abdominal excursions, oxyhemoglobin saturation (pulse oximeter) and body position were recorded simultaneously. Snoring was recorded with a microphone attached in front of the larynx. PSG recordings were performed with a commercially available PSG measurement system for all patients. All EEG recordings were from C3 and C4 electrodes with a sampling rate of 200 Hz. Patients were divided into main and validation datasets with 86 participants in the main dataset: 22 women, age group: 27-84 years, 44 subjects with moderate or severe OSA and 84 in the validation dataset: 28 women, age group 35-75 years, 42 subjects with moderate or severe OSA. The recording parameters (EEG bandpass filter (0.05-200) Hz, sampling rate and EEG channels C2-M1 (left mastoid) and C4-M2 (right mastoid)) were identical in both datasets. All polysomnography (PSG) recordings were performed in a standardized setting between 10 μm and 6 am for each patient.

Sleep stages were assessed visually (manually) according to the guidelines of the American Academy of Sleep Medicine, Inc. Sleep-related respiratory events were assessed visually (manually) according to the updated guidelines of the American Academy of Sleep Medicine. Apnea was detected when the maximum signal deflection decreased by ≥90% from the baseline before the event for ≥10 seconds. Similarly, hypopnea was detected when the maximum signal deflection decreased by ≥30% from the baseline before the event for ≥10 seconds in connection with either ≥3% arterial oxygen desaturation or cortical arousal. Further classification into obstructive, central or mixed respiratory apnea events was based on the simultaneous assessment of the nasal airflow or the thoracic and abdominal deflection.

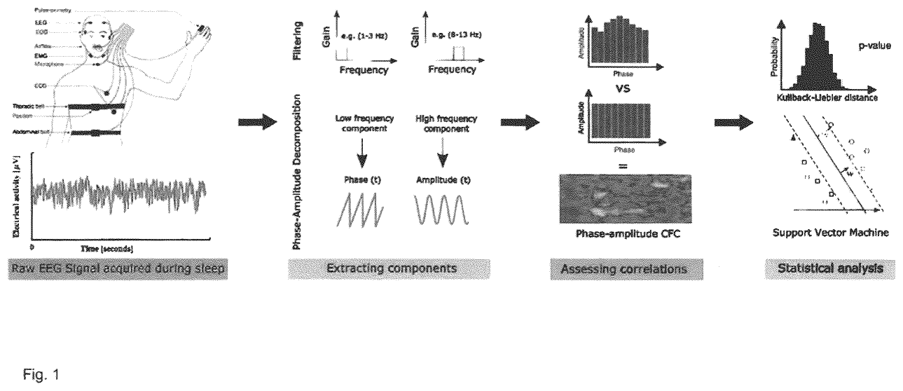

To pre-process the data, raw EEG data were low-pass filtered (fourth-order Butterworth filter; limiting frequency: 100 Hz) to avoid aliasing, followed by high-pass filtering at 0.5 Hz. In order to remove artifacts, the data were subjected to independent component analyses (FastICA) to remove artificial components related to muscle, eye blink, eye movement and line noise artifacts. The pre-processed data were then divided into six different frequency bands, i.e., very low frequencies (VLF, 0.1-1 Hz), delta (1-3 Hz), theta (4-7 Hz), alpha (8-13 Hz), beta (14-30 Hz) and gamma (31-100 Hz) for both electrodes. An overview of the data recording and analysis process is shown in FIG. 1.

The phase-to-amplitude cross-frequency coupling (PACFC) indicates the modulation of the high-frequency power by the low-frequency phase. This CFC modulation index (MI) is then used to identify the phase-amplitude assignment between phase-modulating frequency bands (e.g., for delta) and amplitude-modulating frequency bands (e.g., for alpha). In order to calculate the CFC-MI, the following steps were performed. First, the obtained EEG signal was filtered into two frequency bands, i.e., delta and alpha. After filtering, the Hilbert transform was applied to both the filtered time series to obtain the phase of one time series and the amplitude envelope of the other. This combined time series then has the information in each phase from delta oscillations to amplitude of the alpha rhythm. The possible phase range from −180° to +180° was then divided into 20 units (N) of 18° each and the Kullback-Leibler distance (KL) was calculated using the following formula:

$${D}_{KL} ( P , Q ) = {\sum }_{j \overline{-} 1}^{N} P ( j ) log [ \frac{P ( j )}{Q ( j )} ]$$

D is the KL distance of a discrete distribution P from a distribution Q. The KL distance has the property of always being greater than zero, i.e., D_{KL}(P,Q) ≥0, unless the P and Q distributions are equal, i.e., D_{KL}(P,Q)=0 if P=Q and is similar to the definition of Shannon entropy, which is indicated as follows:

$$H ( P ) = - {\sum }_{j \overline{-} 1}^{N} P ( j ) log [ P ( j ) ]$$

Regarding the Shannon entropy, the KL distance can thus be used to determine the deviation between the distribution of the data and the uniform distribution (U) as follows:

$${D}_{KL} ( P , U ) = log ( N ) - H ( P )$$

Finally, the CFC-MI was calculated for all units, the distribution of the mean amplitude is uniform across all units, indicating no assignment between phase and amplitude. The modulation index (MI) could therefore be calculated as follows:

$$M I = \frac{{D}_{KL} ( P , U )}{log ( N )}$$

Thus, if the mean amplitude is distributed across all phases, the MI would be zero and would be at the maximum if a Dirac delta is obtained in the distribution of the phases. The coupling for the frequency bands theta-gamma and delta-alpha was estimated between the amplitude of higher frequency signals and the phase of lower frequency signals by correlation. The CFC was estimated with a time frame of 5 seconds with an overlap of 50%.

To analyze the significance of these CFC modulation indices, a SVM algorithm (Support Vector Machine) was used to classify different sleep stages based on CFC-MI values from both frequency bands. SVM is an efficient tool for non-linear classification between two datasets that searches for an optimal separating threshold between both datasets by maximizing the margin between the closest points of the classes. Here, the polynomial function kernel was used for this projection due to its good performance and grid search (min. =1, max. =10) to find the few optimal input parameters and gamma (0.25). The selection was checked by a 10-fold cross-validation by using 75% of the data for training and 25% for testing. To validate the efficacy of these CFC modulation indices for clinical applicability, SVM analysis was further applied to predict the clinical scores (RDI and ESS) used in the diagnostic criteria for OSA patients. Here, a Support Vector Regressor (SVR) analysis was performed, which is a multiple regression method based on machine learning, which could assign the observed and trained values and represent the predication accuracy. To obtain the prediction accuracy threshold, an approach based on statistical inference obtained from the Bayesian credible interval was developed. The 75% threshold could distinguish the posterior distribution from the 95% Bayesian credible interval (indicating the inclusion of 95% of the data points). Here, the posterior distribution and credible interval were obtained considering all modulation indices from all sleep stages of both groups and the highest density interval at 95% (range: 0.32-0.89) of the distribution. Thus, the prediction accuracy of over (75%) obtained after 10-fold cross-validation was considered a quite significant result.

To ensure that there were no effects of variables other than the independent variables in the results of the study, scientific controls were performed. It was checked whether the estimated PACFC was independent of the arousals and periodic limb movements of these patients. For this purpose, the arousal index and the periodic limb movement index (PLM) were estimated for each patient, as well as the Pearson correlation between the PACFC in each sleep stage and these two indices. Since previous studies have shown a significant correlation between heart rate variability and clinical OSA scores, it was analyzed whether PACFC is influenced by activity of the autonomous nervous system. For this purpose, the heart rate variability (HRV) was estimated separately for each patient and the Pearson correlation with the PACFC was estimated in each sleep stage. HRV was calculated using the standard deviation of normal-normal intervals; a technique described later. Furthermore, the significance of sample size used in the study was determined. For this purpose, a post-hoc Bayesian posterior distribution analysis was estimated for the MI index of the N1 sleep stage between the two groups.

Of 86 patients analyzed, 42 patients were diagnosed with a respiratory disturbance index (RDI)≤15 per hour (4 with RDI <5 per hour and 38 with RDI between 5 and 15 per hour) and 44 patients were diagnosed with clinically significant OSA (30 patients with RDI between 15 and 30 per hour and 14 patients with RDI >30 per hour). These two groups do not differ significantly in terms of age and gender (p>0.05). The demographic details along with the clinical measures obtained are shown in Table 1. The statistical analysis performed for cross-frequency coupling (CFC) parameters and their association with clinical measures obtained from these two groups yielded significant results, as described below.

TABLE 1
Demographic details of all participants included in the study. Here
RDI: Respiratory Disturbance Index: ESS: Epworth Sleepiness Scale.

| | | | | | | | T-test |
|---|---|---|---|---|---|---|---|
| Dataset | Group | N | Age (years) | Gender | FEI (per hour) | ESS | p-values |
| Main group | FEI ≤ 15 | 42 | 55.67 ± 10.22 | F = 19 | 9.30 ± 3.26 | 10.90 ± 4.53 | Age: 0.746 |
| | | | | M = 23 | | | Gender: 0.085 |
| | FEI > 15 | 44 | 56.52 ± 13.91 | F = 12 | 27.96 ± 12.47 | 11.59 ± 4.45 | RDI: <0.001 |
| | | | | M = 32 | | | ESS: 0.480 |
| Validation | FEI ≤ 15 | 42 | 52.79 ± 9.71 | F = 17 | 11.04 ± 2.97 | 9.86 ± 4.90 | Age: 0.119 |
| group | | | | M = 25 | | | Gender: 0.168 |
| | FEI > 15 | 42 | 56.0 ± 9.02 | F = 11 | 49.48 ± 19.67 | 10.14 ± 5.47 | RDI: <0.001 |
| | | | | M = 31 | | | ESS: 0.817 |

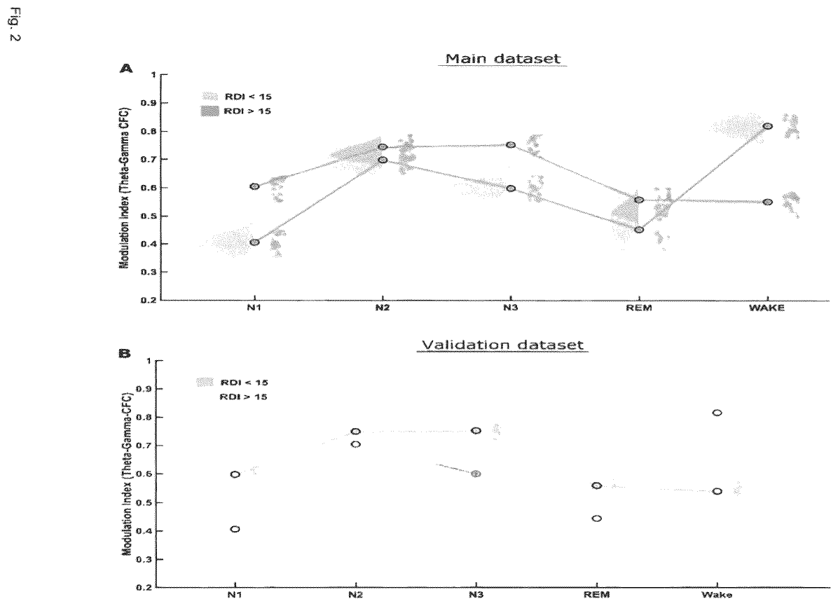

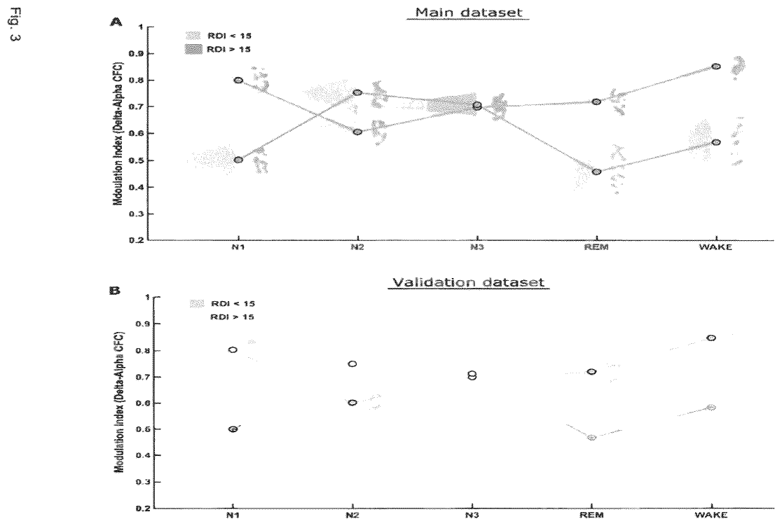

The CFC modulation index (MI) in the theta-gamma frequency bands was significantly reduced (p<0.001) in all sleep stages in patients with clinically significant, i.e., moderate or severe OSA (RDI >15/h), as shown in FIG. 2A. The theta-gamma modulation index was higher during the NREM stages N2 and N3 than during the N1 and REM sleep stages for both groups. The difference in MI values between both groups was highest during N1, was reduced during N2, but increased again during the N3 and REM sleep stages.

A table showing all these values is provided as supplementary Table 1.

FIG. 2 shows the MI differences in theta-gamma CFC, wherein the raincloud plot in FIG. 2a clearly indicates that patients in the RDI >15/h group have a significantly lower theta-gamma CFC modulation index in all sleep stages (NREM and REM) than that of the RDI group ≤15/h, wherein exactly the same pattern was found in both the initial and validation patient groups (FIG. 2b).

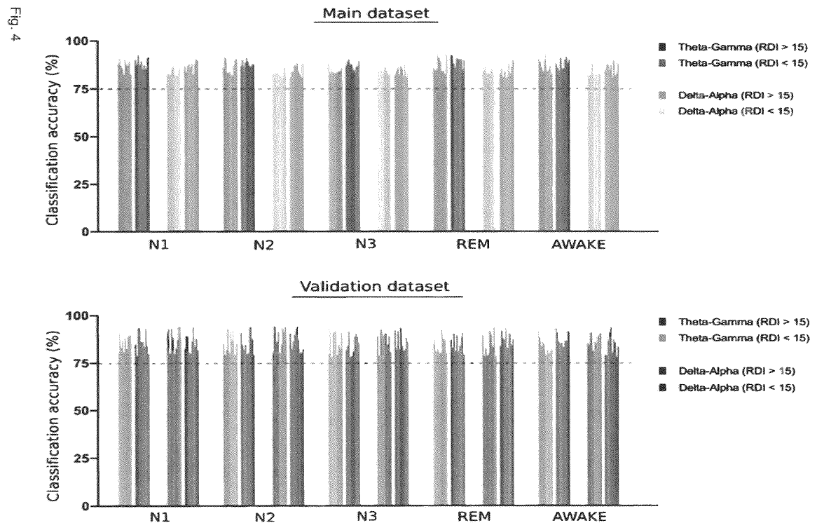

However, CFC-MI at the delta-alpha frequency bands was significantly reduced (p<0.001) only during REM and N1, but not in the N3 sleep stage in patients with clinically significant OSA compared to patients with mild OSA or no OSA (RDI≤15/h). as shown in FIG. 3A. Furthermore, CFC-MI in NREM sleep stage N2 was higher in patients with significant OSA (i.e., RDI >15/h) compared to patients without OSA (RDI≤15/h). (See Table 1).

FIG. 3 shows the MI differences in delta-alpha CFC, wherein the raincloud plot in FIG. 3a indicates that patients in the RDI >15/h group have a significantly lower delta-alpha CFC modulation index than in the REM and N1 sleep stages and a significantly higher MI in the N2 sleep stage than patients in the RDI≤15/h group. The delta-alpha CFC modulation index in the NREM-3 (N3) sleep stage is almost identical in both patient groups. It is notable that exactly the same pattern was found in both the initial and validation datasets (FIG. 3b).

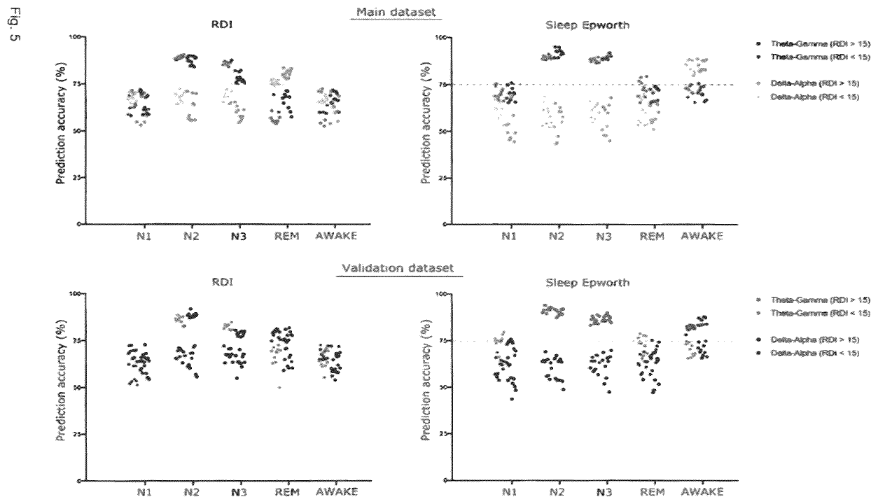

The SVM analysis (Support Vector Machine) showed a significant classification of all four sleep stages and the waking stage using CFC modulation indices separately from the theta-gamma and delta-alpha frequency bands. The overall classification accuracy was higher than 80% and reached up to 94% for the classification of the waking stage using theta-gamma CFC-MI, as shown in FIG. 4.

FIG. 4 shows the SVM classification of different sleep stages. The bar chart shows the classification accuracy of the Support Vector Machine (SVM) of different sleep stages using theta-gamma and delta-alpha cross-frequency coupling modulation indices (CFC). The group of 10 bars in each set represents the accuracy obtained for 10-fold cross-validation. A dotted line is indicated at 75% accuracy to emphasize the significant level of classification obtained for all CFC metrics used in the study. All accuracy values are shown in the supplementary Table 1.

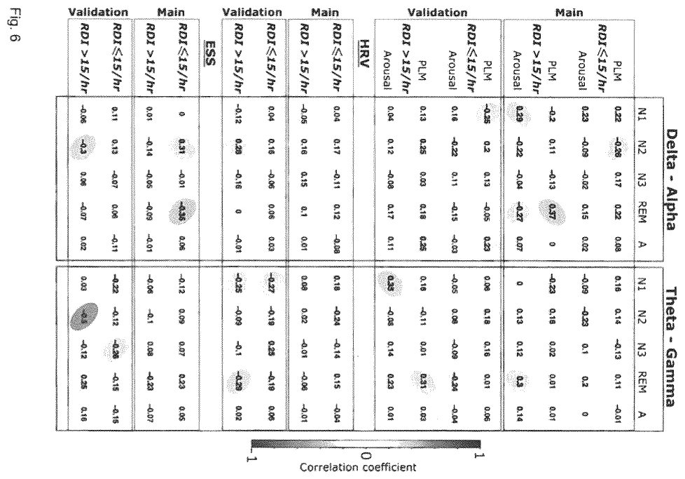

Furthermore, SVM was able to predict RDI and Epworth Sleepiness Scale (ESS) in different sleep stage with significant accuracy (more than 75%) using the CFC-Mi in both frequency band pairs. The theta-gamma CFC was able to significantly predict the RDI und ESS in NREM sleep stages (N2 and N3). Delta-alpha CFC in REM sleep stages was able to significantly predict RDI, and delta-alpha CFC in waking stages was able to significantly predict ESS. The details of all predictions are shown in FIG. 5.

FIG. 5 shows the prediction of RDI and ESS. The scatter plot shows the prediction accuracy of the Support Vector Machine (SVM) of the Respiratory Disturbance Index (RDI) and the Epworth Sleepiness Scale (ESS) using the modulation indices from theta-gamma and delta-alpha cross-frequency coupling (CFC). The group of 10 points in each set represents the accuracy obtained for 10-fold cross-validation. An accuracy over 75% was obtained for significant; illustrated by a dotted line in the plot.

Among the 84 patients analyzed from this dataset, 42 patients were diagnosed with a Respiratory Disturbance Index (RDI)≤15 per hour and 42 patients were diagnosed with clinically significant OSA (3 patients with RDI between 15 and 30 per hour and 39 patients with RDI >30 per hour). In this dataset, too, both groups did not differ significantly in terms of age and gender (p>0.05). The demographic details are shown in Table 1.

The statistical analysis performed for this dataset yielded quite similar results to the first (main) dataset and thus confirmed most of the results. CFC-MI at the theta-gamma frequency bands were also reduced in clinically significant OSA patients (RDI >15/h), similar to the main findings that showed higher modulation also during NREM-N2 and N3 sleep stages (FIG. 2B). Similarly, CFC-MI in delta-alpha frequency bands was also significantly reduced only during REM and N1, but not in N2 and N3 sleep stages in patients with clinically significant OSA, as can be found in the main dataset (FIG. 3B).

The SVM analysis for the classification of sleep stages using CFC modulation indices for theta-gamma and delta-alpha frequency bands showed replicable results using the validation dataset with a classification accuracy of over 80%, as shown in FIG. 4.

Similarly, the validation dataset was further able to replicate the prediction results for clinical parameters-RDI and Epworth Sleepiness Score (ESS)—with an accuracy of more than 75%, wherein the same CFC modulation indices as in the main dataset were used, as shown in FIG. 5.

However, the correlation between the Epworth Sleepiness Score and PACFC was also not significant in any sleep stage (FIG. 6).

No significant correlation (all p>0.05) was found between the arousal and PLM indices to PACFC in any sleep stage (FIG. 6). Furthermore, no significant correlation was found between heart rate variability and PACFC in any sleep stage, which showed no influence of the autonomous nervous system on PACFC (FIG. 6).

FIG. 6 shows the correlation of clinical parameters with CFC measurements. FIG. 6a shows the correlation coefficients between arousal and periodic limb movement indices (PLM) to phase-amplitude cross-frequency coupling (PACFC) for each sleep stage separately. For both delta-alpha and theta-gamma, PACFC are separated by the columns. FIG. 6b shows the correlation coefficients between heart rate variability (HRV) and PACFC in different sleep stages separated by the columns. FIG. 6c shows the correlation coefficients between the Epworth Sleepiness Score (ESS) and PACFC in different sleep stages. The correlation for the main and validation groups is indicated separately in each row. The r-values are indicated for the correlation. All correlations were not significant (p>0.05).

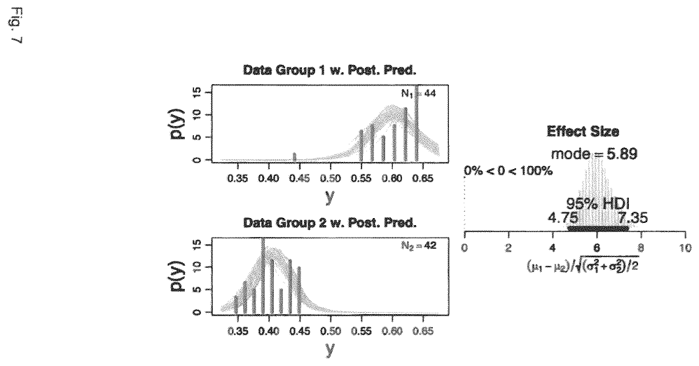

The Bayesian posterior distribution showed that the 95% High Density Interval (HDI) is within the obtained effect in the analyzed data (FIG. 7), indicating a sufficient sample size for the primary result in this study.

FIG. 7 shows the posterior distribution of the analyzed groups. The plot on the right shows the distribution histogram of the effect size indicating the 95% High Density Interval (HDI) found in the analyzed data. This indicates a sufficient sample size of the included subjects based on the primary result (i.e., phase-amplitude cross-frequency coupling for theta-gamma in N1 sleep stage). The plots on the left show the probability distribution with superimposed posterior predictive distribution of the raw data for each data sample.

A significant reduction of the theta-gamma modulation index (MI) was found in the central sensorimotor cortical regions in patients with moderate or severe OSA compared to patients with mild OSA or healthy subjects. MI reduction during sleep was frequency band-specific; it included theta-gamma connectivity during all sleep stages, while delta-alpha connectivity only during REM and N1. Therefore, a global reduction in modulation (both theta-gamma and delta-alpha) was observed during REM and N1. Furthermore, MI differences between stages were so distinctive that the classification of sleep stages based on MI values was achieved in both datasets in both patient groups. Furthermore, theta-gamma MI during N2 and N3 very reliably predicted both RDI and ESS and delta-alpha Mi very reliably predicted RDI during REM.

These novel results, which show the functional separation between theta and gamma activity in the cortical sensorimotor area during all sleep stages in OSA patients, have pathophysiological and clinical implications that need to be further discussed.

Theta-gamma PACFC has been associated to motor, sensory and cognitive processes. In individuals with RDI≤15/h, there is a very strong coupling between theta and gamma oscillations in both the N2 and N3 stages. In moderate and severe OSA, the modulation index decreases quite significantly during N3, while it decreases to a much lower extent in N2. During short waking periods between sleep stages, the MI increased significantly in patients with moderate/severe (i.e., significant) OSA compared to patients with RDI≤15/h. This can reflect a physiological (motor, respiratory, cognitive) compensatory role for cortical arousals and/or intermittent waking periods in order to promote a transient increase in central sensorimotor connectivity in patients with significant OSA. A synaptic net potentiation associated with wakefulness can provide the basis for such an increase in connectivity.

The recorded neuronal activity of the sensorimotor cortex can either be primarily generated in this anatomical area or be an epiphenomenon of activity from other subcortical/thalamic subcortical/thalamic or neural brainstem master generators. These generators promote neuromodulation of the cranial neural pathways, leading to a reduced muscle tonus of the upper airways associated with upper airway obstructions that occur during respiratory events in sleep in OSA.

In patients with focal epileptic seizures, the intensity of the theta-gamma phase-amplitude coupling during sleep was the highest during N3 and the lowest during REM. In patients with moderate or severe OSA, theta-gamma PACFC was the highest during N2 (see FIG. 2). The coupling of fast and slow oscillations was significantly reduced during REM compared to N2 and N3 in all OSA patient groups; this reduction was more distinctive in patients with significant OSA. Strong CFC between high-frequency and slow-wave oscillations during slow-wave sleep was found in hippocampus of anaesthetized primates. EEG measures the summed postsynaptic potentials of synchronously active regions of cortex and hippocampus that have spread across brain, skull and scalp. Although they often coincide, it is not necessary that the firing of action potentials is related to oscillations of postsynaptic potentials. Therefore, the oscillations of postsynaptic potential do not always lead to the firing . . . of postsynaptic action potentials. It is difficult to distinguish cortical from hippocampal output in humans on the basis of surface EEG recordings alone.

A significant increase in delta-alpha CFC-MI is observed during N2 in patients with significant OSA compared to patients with RDI≤15/h. This finding can represent compensatory increased activity of the sensorimotor cortex during the respiratory event Rich-N2 stage to exert better motor control of breathing in more severely affected OSA patients (RDI>15/h).

Delta-band oscillation in spike and local field potentials, activity in the somatosensory whisker barrel cortex of awake mice is phase-bound to respiration. Thus, the respiratory activity directly modulates slow (1-4 Hz) rhythmic neuronal activity in somatosensory whisker barrel cortex and indirectly modulates gamma band power through phase-amplitude coupling mechanisms in mice. Our results provide preliminary evidence for a physiological involvement of delta and gamma band oscillations in the control of respiration in humans, particularly in OSA, and should be further validated.

Particularly, the delta-alpha CFC-MI remains quite stable during N3, regardless of OSA severity. Thus, delta-alpha coupling can be involved in brain connectivity processes, which remain stable during N3, or it can also be a surrogate marker for known respiratory stability during N3, when apneas and hypopneas occur less often.

Given that both theta-gamma and delta-alpha modulation indices can reliably predict the classification of sleep stages according to AASM criteria, it can be assumed that quite different sleep stage-specific PACFC patterns with the above-mentioned frequency bands exist. These different patterns are apparently quite robust, involve at least the two oscillatory channels mentioned above (gamma-theta, delta-alpha) and can include other oscillatory channels. Since a major part of the datasets tested belongs to patients with RDI>15/h. these sleep stage-specific coupling patterns seem to remain quite robust regardless of the degree of the accompanying sleep-related dyspnea.

The global (theta-gamma and delta-alpha) connectivity reduction, as represented by MI reduction, during REM in the central sensorimotor areas in OSA patients with RDI>15/h compared to patients with RDI≤15/h can provide a surrogate marker for reduced central motor power during REM. This reduced motor power probably affects many muscle groups and particularly affects the muscles that control upper airway patency, as they correlate strongly with RDI. The MI difference is particularly distinctive in delta-alpha CFC-MI (FIG. 2). Differential modulation of global and local oscillations during REM sleep has been reported.

Thus, multifrequency (global) MI in sensorimotor areas during REM can serve as a surrogate marker for OSA disease severity. In support of this argument, further analysis (FIG. 4) showed that delta-alpha MI during REM very reliably predicted the mean RDI in both datasets tested.

Theta-gamma MI during N2 and N3 and delta-alpha MI during short waking periods from sleep prove to be reliable surrogate markers for patient-reported excessive daytime sleepiness (EDS). These results also point to possible sleep-related oscillatory and stage-specific physiological mechanisms that promote attentiveness and alertness in humans. Subjective assessments of sleepiness have shown distinctive associations with increased functional connectivity in widespread regions within the sensorimotor network.

The phase-amplitude cross-frequency coupling (PACFC) modulation index was the primary final result and was tested as predictor for clinical variables in this study. Regarding its significance: spatial working memory performance, maintenance of working memory with multiple items, changes in perceptual results, learning, visual attention and perception are some of the functional features that have been associated with PACFC modulation. Furthermore, it has also been shown that it contributes to BOLD connectivity (dependent on blood oxygen levels) and has connections to brain alterations that occur in several neurological disorders such as epilepsy, Parkinson's disease, Alzheimer's disease, schizophrenia, obsessive-compulsive disorder (COD) and minimal cognitive impairments (MCI). Although there is sufficient evidence that PACFC is potentially a promising approach to decipher brain function and some of its pathologies with a credible physiological mechanism (the low-frequency phase reflects local neuronal excitability, while high-frequency power increases reflect either a general increase in population synaptic activity or selective activation of a connected neuronal subnetwork). There are still some unanswered questions about the origin, causality and mechanism of these oscillations. The choice of modulation index (MI) in this study is based on the knowledge that MI has proven to be the most robust to confounding influences of moderators among some of the most commonly used phase-amplitude coupling measurements, including data length, signal-to-noise ratio, and sampling rate when approaching Nyquist frequencies.

The above-mentioned evidence can open new possibilities for intervention by means of pharmacological or transcranial magnet stimulation (TMS) of the sensorimotor cortex in OSA patients. TMS during sleep has been applied to the corticomotor-somatotopic representation of the tongue; induced twitches have briefly improved airflow without causing arousals in OSA patients. However, the effect on other motor areas and the neurocognitive effect of TMS has not been extensively studied in OSA. The finding that the above-mentioned CFC modulation indices In both frequency bands could significantly predict the RDI and Epworth Sleepiness Score (ESS) in OSA patients should be further validated in larger studies.

The result that 1) theta-gamma CFC-MI significantly predicted RDI and ESS in NREM (N2, N3), 2) delta-alpha CFC-MI predicted RDI in REM, and 3) delta-alpha CFC-MI significantly predicted ESS during wakefulness suggests that the CFC-MI theta-gamma and delta-alpha metrics can represent completely different processes in human sleep physiology. Delta-alpha coupling seems to be significant 1) for the control of upper airway motor stability and respiration during REM sleep and 2) for the control of attention and alertness processes, as represented by ESS, during (cortical) arousal and short waking periods between sleep stages. Theta-gamma phase-amplitude coupling seems to be very significant 1) for the control of upper airway stability and respiration during NREM N2 and N3 sleep and 2) for attention- and alertness-related processes (represented by ESS), which occur during N2 and N3 sleep. These results suggest that the cortical central sensorimotor regions can actually be an important hub in the networks regulating sleep and/or sleep-related respiratory activity. After validation in larger patient cohorts, the modulation index could eventually be integrated as an additional metric representing both the severity of dyspnea and daytime sleepiness in patients with OSA.

The replication of the results in the validation dataset further supports the reproducibility and validity of the results and indicates their clinical significance as diagnostic surrogate markers. Furthermore, the scientific control results clearly showed no influence of arousal, periodic limb movement and the autonomous nervous system in the PACFC measurement.

It is suggested that theta-gamma MI at the sensorimotor cortical areas during N2 and N3 and delta-alpha CFC-MI at the sensorimotor cortical areas during REM can be used as a metric of dyspnea during sleep in humans and thus as measure of OSA severity. Therefore, further analyzes of theta-gamma FCKW during N2 and N3 and delta-alpha FCKW during REM should be performed. Calculation of these Mis in further cortical areas can provide additional insight into OSA pathogenesis and diagnosis. Neurophysiological and neuroimaging studies on thalamocortical connectivity based on the present results can further clarify mechanisms of excessive daytime sleepiness. Furthermore, it would be interesting to assess the effect of established evidence-based therapies for OSA, such as positive airway pressure (PAP) therapy, on PACFC.

Functional separation of the central cortical sensorimotor area between theta and gamma activity is observed in all sleep stages of OSA. Further significant delta-alpha sensorimotor area separation occurs during REM and N1 stages in OSA. Thus, sensorimotor separation is widespread, shows frequency band- and sleep stage-specific patterns, and provides further evidence for the presence of central sensorimotor dysfunction in OSA patients. The theta-gamma modulation index during N2 and N3 reliably predicts patient-reported sleepiness. Therefore, modulation indices can be used as surrogate diagnostic predictive markers for dyspnea during sleep and for patient-reported excessive daytime sleepiness.

In summary, the method described is suitable for determining the severity of obstructive sleep apnea and accompanying daytime sleepiness, wherein the method comprises the following steps:
recording two body function data, wherein both body function data are EEG measurement signals of an electroencephalography at the electroencephalography points C3 and c4.
dividing the measurement signals into frequency bands, wherein the division is carried out separately for each measurement point,
determining the cross-frequency modulation indices,
determining the Respiratory Disturbance Index and daytime sleepiness using a Support Vector Machine based on cross-frequency modulation indices.

The Respiratory Disturbance Index and daytime sleepiness are a measure of the severity of obstructive sleep apnea and its accompanying symptoms.

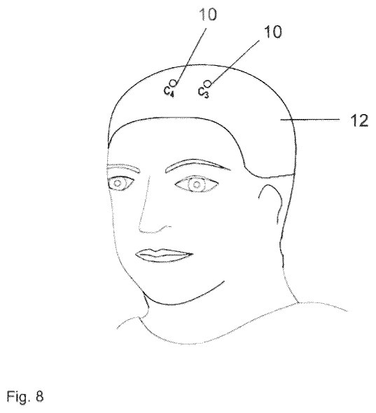

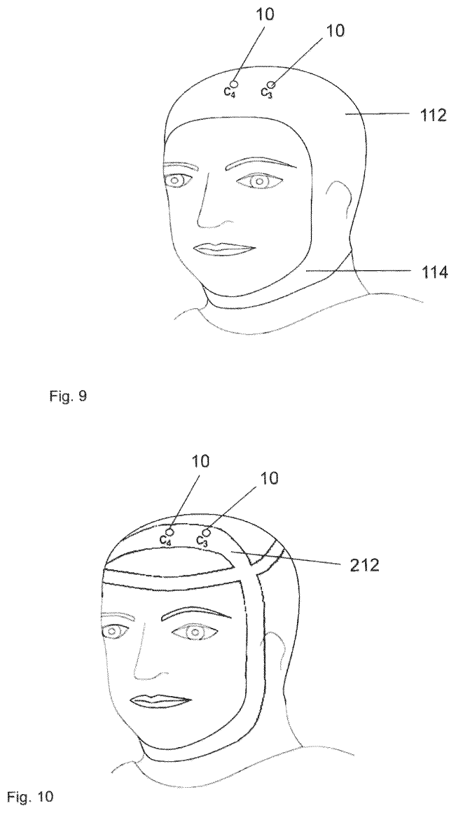

FIGS. 8 to 10 show embodiments of a measurement device for detecting the measurement signals C3 and C4. These measurements devices are part of a device, not shown, for carrying out the method described. The measurement devices from FIGS. 8 and 9 are a headgear in which two sensors 10 for detecting the C3 and C4 measurement signals are incorporated. The headgear is designed here as a cap 12 (FIG. 8) or as a cap 112 with a chin part 114 (FIG. 9). Alternatively, interconnected bands 212 can also be provided as headgear, which are attached to the head and in which two sensors 10 for detecting the C3 and C4 measurement signals are incorporated (see FIG. 10).

1. A method for determining a measure of the degree of an obstructive sleep apnea and/or its consequence by means of the following steps:
a. defining a measure of the degree of an obstructive sleep apnea and/or its consequence,
b. providing two EEG measurement signals of an electroencephalography at the electroencephalography points of a 10-20 international EEG system,
c. dividing the BEG measurement signals into frequency bands,
d. determining at least one cross-frequency modulation index using data from at least two different frequency bands,
e. determining the measure of the degree of an obstructive sleep apnea and/or its consequence by means of the at least one cross-frequency modulation index.

2. The method according to claim 1, characterized in that the EEG measurement signals are determined during sleep in the laboratory or at home.

3. The method according to claim 1, characterized in that the measure of the degree of an obstructive sleep apnea and/or its consequence, particularly the Respiratory Disturbance Index and/or daytime sleepiness, is determined only on the basis of the two EEG measurement signals.

4. The method according to claim 1, characterized in that the electroencephalography points at which the measurement signals are recorded are points C3 and C4.

5. The method according to claim 1, characterized in that the measurement signals are divided into frequency bands separately for each measurement point.

6. The method according to claim 1, characterized in that the at least one cross-frequency modulation index is determined by means of a phase-amplitude cross-frequency coupling.

7. The method according to claim 1, characterized in that the measurement signals are divided into at least two of the following frequency bands: low frequency band from 0.1 to 1 Hz, delta band (1 to 3 Hz), theta band (4 to 7 Hz), alpha band (8-13 Hz), beta band (14 to 30 Hz) and gamma band (31-100 Hz).

8. The method according to claim 6, characterized in that the measurement signals are divided into the two frequency bands alpha band (8-13 Hz) and delta band (1 to 3 Hz) to determine a phase-amplitude cross-frequency coupling, wherein the amplitude envelope is preferably determined from the alpha band and the phase of the measurement signals is determined from the delta band by means of the phase-amplitude cross-frequency coupling.

9. The method according to claim 6, characterized in that the measurement signals are divided into the two frequency bands theta band (4 to 7 Hz) and gamma band (31-100 Hz) to determine a phase-amplitude cross-frequency coupling, wherein the amplitude envelope is preferably determined from the gamma band and the phase of the measurement signals is determined from the theta band by means of the phase-amplitude cross-frequency coupling.

10. The method according to claim 1, characterized in that a correlation database with correlation data is provided between the at least one cross-frequency modulation index and the measure of the degree of an obstructive sleep apnea and/or its consequence and in that the measure of the degree of an obstructive sleep apnea and/or its consequence is determined from the at least one cross-frequency modulation index by means of the data of the correlation database.

11. The method according to claim 1, characterized in that the measure of the degree of an obstructive sleep apnea and/or its consequence, particularly the Respiratory Disturbance Index and/or daytime sleepiness, is determined using a Support Vector Machine based on the at least one cross-frequency modulation index.

12. The method according to claim 1, characterized in that the measurement signals are recorded during sleep and the sleep is divided in sleep stages, wherein the at least one cross-frequency modulation index is determined depending on the sleep stages.

13. The method according to claim 1, characterized in that all method steps are carried out automatically.

14. A device for carrying out a method for determining a measure of the degree of an obstructive sleep apnea and/or its consequence, in particular a device for carrying out a method according to claim 1, characterized in that the device comprises a headgear with only two sensors for determining the EEG signals, wherein the sensors are preferably arranged in a way that the measurement signals are recorded at the points C3 and C4.

15. The device according to claim 14, characterized in that in that the headgear is a headband, a cap or a beanie.
